(19) United States
(12) Patent Application Publication
(10) Pub. No.: US 2017/0100476 A1
(43) Pub. Date: Apr. 13, 2017

(54) HSP-FREE ALLERGEN PREPARATION

(71) Applicant: ASIT BIOTECH S.A., Brussels (BE)

(72) Inventors: Thierry Legon, Bierbeek (BE); Sabine Pirotton, Brussels (BE); Gael Placier, Liége (BE)

(21) Appl. No.: 15/128,780

(22) PCT Filed: Apr. 9, 2015
(86) PCT No.: PCT/EP2015/057772

§ 371 Date: Sep. 23, 2016

(30) Foreign Application Priority Data
Apr. 10, 2014 (EP) 14164293.4

(51) Int. Cl. A61K 39/36; A61K 9/00; A61K 47/26; A61K 39/35

(52) U.S. Cl. CPC A61K 39/36; A61K 39/35; A61K 9/0019; A61K 47/26; A61K 2039/55511

(57) ABSTRACT

A pharmaceutical preparation comprising—10 to 200 μg/ml of fragments of an antigenic structure which induces allergic reaction—2 to 6% (w/v) mannitol—0.5 to 2% (w/v) trehalose—water, wherein said preparation essentially does not comprise heat shock proteins.

The present invention relates to a pharmaceutical preparation, especially useful for treating allergy, autoimmune disease or graft rejection.

U.S. Pat. No. 6,312,711 discloses a pharmaceutically or food composition intended for treating pathologies associated with graft rejection or allergic autoimmune reaction comprising the administration of a complex of a stress protein and epitopes of an antigenic structure.

WO 2013/011095 discloses a pharmaceutical preparation for subcutaneous injection comprising between 0.5 ng and 200 μg of HSP70 between 0.5 and 100 μg of fragments of an antigenic structure.

There has been a lot of research in connection with the inclusion of heat shock proteins in pharmaceutical preparations for tolerance induction, but clinical outcome is not always satisfying. There is still a need for an improvement of these preparations.

Surprisingly it has now been found that a pharmaceutical preparation comprising
10 to 200 μg/ml of fragments of an antigenic structure which induces allergic reaction
2 to 6% (w/v) mannitol
0.5 to 2% (w/v) trehalose
water,
   wherein said preparation essentially does not comprise heat shock proteins, may be used for a safe treatment with a high efficiency.

The fragments of the antigenic structure dissolved in a solution comprising mannitol and trehalose, but without the addition of heat shock proteins are on the one hand safe in administration, but on the other hand also suitable for the treatment for inducing tolerance to the related antigen.

Mannitol and trehalose have been used in prior art for the formulation of pharmaceutical preparations, but typically in the context with lyophilized products.

The pharmaceutical preparation of the present invention is not lyophilized during production, but nevertheless provides advanced stability upon storage.

The pharmaceutical preparation comprises about 2 to 6% (w/v) mannitol. The suitable amount of trehalose is in an amount of 0.5 to 2% (w/v), wherein the volume is measured at 25° C.

The preparation comprises also a buffering agent, a phosphate buffer is preferred.

In one embodiment of the invention, the pharmaceutical preparation is in a form for subcutaneous injection.

The preparation of the invention is essentially free of heat shock proteins. Essentially free of heat shock proteins refers to concentration of less than 1 μg/ml, preferably less than 1.0 ng/ml, more preferably less than 0.5 ng/ml.

The fragments of the antigenic structure are preferably prepared by enzymatic hydrolysis of the antigenic structure. Preferred forms for preparing fragments of antigenic structures. Preferred ways of obtaining hydrolyzed allergen fragments preferably free of non-protein components of the antigens are the methods described in WO 2008/000783, WO 2009/083589 and WO 2012/172037; these methods are incorporated by reference. The major steps of these methods are:
an extraction of allergenic proteins from the source of allergens;
a first purification step followed by a denaturation preferably with reducing and chaotropic agents
a further purification step
hydrolysis of the protein.

A further denaturing step may be used prior to hydrolysis.

The antigenic structures are selected from antigenic structures which induce allergic reaction. Such antigenic structures which are also referred to as allergens. Preferred allergens are natural protein allergens. Suitable examples are selected from milk allergens, venom allergens, egg allergens, weed allergens, grass allergens, grass pollen antigens, tree allergens, shrub allergens, flower allergens, grain allergens, fungi allergens, fruit allergens, berry allergens, nut allergens, seed allergens, bean allergens fish allergens, shellfish allergens, meat allergens, spices allergens, insect allergens, mite allergens, animal allergens, animal dander allergens, allergens of Hevea brasiliensis. Very preferred allergens are grass pollen allergens, peanut allergens, house dust mite allergens, ragweed allergens and Japanese cedar allergens.

In one embodiment of the invention, the pharmaceutical preparation is used in a treatment comprising at least two injections in a patient at different time points, preferably wherein the preparation is for use in a treatment comprising of 2 to 20 injections with increasing amounts of the preparation.

A further embodiment of the present invention is a vial or application device comprising 0.2 to 1.50 ml or 0.5 to 1.50 ml of the pharmaceuticals preparation of the invention.

A further embodiment of the invention is a kit comprising 2 to 20 or 2 to 30 vials or application devices, said application devices comprising the necessary amount of the pharmaceutical preparation of the invention for use in a treatment comprising injections with increasing amounts of the preparation.

The application device is a more convenient form because it avoids the diffusion of the active ingredients present on the surface of the needle in the derma.

In a typical embodiment, the application devices are syringes. For example, the application devices could comprise a solution of 100 μg/ml of the preparation and the first syringe could comprise 50 μl, other devices 100, 200, 500 μl and 1000 μl. The advantage is that the preparation is ready to use. Prefilled application devices reduce the error rate during application.

Surprisingly the preparation of the present invention is stable at the temperature of a refrigerator for at least six months, preferably more than a year. Even if stored at room temperature, stability allows storage for a similar time period. These properties avoid cumbersome work related to dissolving lyophilized preparations prior to use.

It is believed that mannitol and trehalose provide for the high stability of the preparation, thus, increasing the safety and efficacy of the preparation.

A further embodiment of the present invention is a method for treating allergy comprising administering to a patient by a subcutaneous injection, a cumulated dose of 40 to 1000 μg of fragments of an antigenic structure using the pharmaceutical preparation of the invention.

Preferred time intervals between injection sessions are 2 to 10 days.

In one preferred embodiment, the patient receives two subcutaneous injections at different loci of the patient's body during the injection session, e.g. doctor visit. The injections are preferably performed with a 30 to 60 min interval. Preferred loci for injections are the left and the right arm of a patient.

### EXAMPLES

The safety and effect of the preparation was analyzed. The study was conducted in the University Hospital Carl-Gustav-Carus, Dresden, Germany together with the University Hospital of Cologne, Cologne, Germany.

### Effect

As test material, an allergen extract from Lolium perenne was used comprising:
100 μg/ml hydrolyzed pollen allergen extracts prepared according to WO 2008/000783
42 mg/ml mannitol
10.2 mg/ml trehalose
0.69 mg/ml phosphate
0.70 mg/ml NaCl
and water.

The administration scheme included 6 visits with 2 injections administered at a time interval of 30 min in each arm. Within the total of 12 subcutaneous injections on 6 visits, the cumulative dose was 490 μg. Before and after treatment, additional visits (visit 1, visit 8) were conducted. It was a monocentric study with 61 patients with allergic rhinitis or rhinoconjunctivitis due to grass pollen allergens. In this analysis, 44 patients were included, the others are still within the treatment.

The dosage regimen was 5 μg, 10 μg, 20 μg, 40 μg, 70 μg and 100 μg at a concentration of 100 μg/ml. As it is applied in each arm, the double amount was applied per visit. From this interim analysis of 44 patients, 38 completed the study, 6 drop-outs occurred, one was for systemic reactions, one was for private reasons and 4 patients were no longer reachable.

Including the drop-outs, the cumulative dose applied to the patients can be derived from table 1.

TABLE 1

| Cumulative dose [μg] | Frequency | Percentage |
|---|---|---|
| 30 | 1 | 2.3 |
| 60 | 1 | 2.3 |
| 70 | 1 | 2.3 |
| 150 | 1 | 2.3 |
| 190 | 2 | 4.5 |
| 290 | 2 | 4.5 |
| 390 | 1 | 2.3 |
| 490 | 35 | 79.5 |
| Total | 44 | 100.0 |

Prior to the treatment, a skin prick test was conducted. The results of the skin reaction (expressed in mm) can be seen in table 2. The positive control was an injection of histamine.

TABLE 2

| | | | Grass | | Dust | Dust | | |
|---|---|---|---|---|---|---|---|---|
| | Negative | Positive | Pollen | Ragweed | mite DP* | mite DF* | Cat | Dog |
| | Control | Control | Allergens | Allergens | Allergens | Allergens | Allergens | Allergens |
| N | 44 | 44 | 44 | 44 | 44 | 44 | 44 | 44 |
| Mean | 0 | 6.23 | 6.66 | 0.34 | 1.36 | 0.70 | 1.95 | 1.52 |
| Median | 0 | 6.00 | 6.00 | 0 | 0 | 0 | 0 | 0 |
| Standard | 0 | 1.70 | 2.37 | 0.96 | 2.25 | 1.67 | 2.65 | 2.03 |
| Deviation | | | | | | | | |
| Minimum | 0 | 4 | 4 | 0 | 0 | 0 | 0 | 0 |
| Maximum | 0 | 13 | 15 | 3 | 9 | 8 | 9 | 7 |

DP: Dermatophagoïdes pteronyssinus
DF: Dermatophagoïdes farinae

Additionally, the serum level of IgE was tested. The results are reported in table 3.

TABLE 3

| | IgE [kU/l] |
|---|---|
| | Lolch Weidelgras g5 |
| Mean | 43.07 |
| Standard deviation | 33.94 |
| 25^{th} percentile | 13.70 |
| Median | 29.10 |
| 75^{th} percentile | 62.70 |
| Minimum | 2.4 |
| Maximum | 101.0 |

The efficacy of the experiment was tested using a diagnostic solution from ALK-Abel\grave{l} named “Provokationslösung ALK-lyophilisiert Gräser”. Increasing amounts were injected and the reaction was tested in a conjunctival provocation test (CPT) at visit 1, visit 6 and visit 8. The CPT stages were defined as in table 4 (Gronemeyer's grading, Richelman et al 2003 Arch. Allergy. Immunol. 130; 51-59).

TABLE 4

| Stage | Findings |
|---|---|
| 0 | No subjective or visible reaction |
| I | Itching, reddening, foreign body sensation |
| II | Stage I and in addition tearing, vasodilatation of |
| | conjunctiva, bulbi |
| III | Stage II and in addition vasodilatation and erythema |
| | of conjunctiva tarsi, blepharospasm |
| IV | Stage III and in addition chemosis, lid swelling |

The results are reported in Tables 5 to 7.

TABLE 5

| | | Percentage of |
|---|---|---|
| Visit 1 | Rate | valid |
| Valid | 37 | 100 |
| No response | 1 | 2.7 |
| Reaction at 100 SQ-E/ml | 8 | 21.6 |
| Reaction at 1,000 SQ-E/ml | 18 | 48.6 |
| Reaction at 10,000 SQ-E/ml | 10 | 27 |
| Missing | 7 | |
| Total | 44 | |

TABLE 6

| | | Percentage of |
|---|---|---|
| Visit 6 | Rate | valid |
| Valid | 38 | 100 |
| No response | 29 | 76.3 |
| Reaction at 100 SQ-E/ml | 2 | 5.3 |
| Reaction at 1,000 SQ-E/ml | 2 | 5.3 |
| Reaction at 10,000 SQ-E/ml | 5 | 13.2 |
| Missing | 6 | |
| Total | 44 | |

TABLE 7

| | | Percentage of |
|---|---|---|
| Visit 8 | Rate | valid |
| Valid | 37 | 100 |
| No response | 24 | 64.9 |
| Reaction at 100 SQ-E/ml | 0 | 0 |
| Reaction at 1,000 SQ-E/ml | 1 | 2.7 |
| Reaction at 10,000 SQ-E/ml | 12 | 32.4 |
| Missing | 7 | |
| Total | 44 | |

In summary, only one patient (2.7%) was showing no response to the “Provokationslösung” prior to treatment. This improved to 29 patients (76.3%) during treatment and 24 patients (64.9%) after treatment. An improvement of the CPT is achieved at visit 8 for 93.8% of all patients.

### Safety

24 patients did not mention any adverse effect, 20 patients had one or more adverse effects, but no one experienced a serious adverse effect. At the site of injection, typically a redness and wheal occurs at injection site. The size wheal is an important safety indicator. It is measured about 30 minutes after injection.

Table 8 shows the variation of the redness diameter in cm during the visits and injections. Surprisingly and unexpected, the typical increase of redness diameter with increasing amounts of injection did not occur. In contrast, the redness diameter was almost stable or even decreased slightly during treatment. This is unexpected and an indication of the well-tolerated administration.

TABLE 8

| | Mean value of the redness (cm) | | | | | | | | | | | |
|---|---|---|---|---|---|---|---|---|---|---|---|---|
| | Visit 2 | | Visit 3 | | Visit 4 | | Visit 5 | | Visit 6 | | Visit 7 | |
| | Inj. | Inj. | Inj. | Inj. | Inj. | Inj. | Inj. | Inj. | Inj. | Inj. | Inj. | Inj. |
| | 1 | 2 | 3 | 4 | 5 | 6 | 7 | 8 | 9 | 10 | 11 | 12 |
| Valid | 44 | 44 | 44 | 44 | 43 | 42 | 42 | 42 | 40 | 40 | 37 | 36 |
| Missing | 0 | 0 | 0 | 0 | 1 | 2 | 2 | 2 | 4 | 4 | 7 | 8 |
| Mean | 1.69 | 1.92 | 2.46 | 2.54 | 1.91 | 1.98 | 2.01 | 2.11 | 2.09 | 2.09 | 1.70 | 1.85 |
| Standard | 1.40 | 1.67 | 1.21 | 1.28 | 1.25 | 1.09 | 1.12 | 1.22 | 1.76 | 1.58 | 1.65 | 1.75 |
| deviation | | | | | | | | | | | | |
| 25^{th} | 0 | 0.4 | 1.63 | 1.63 | 1.0 | 1.0 | 1.38 | 1.0 | 0 | 1.0 | 0 | 0 |
| percentile | | | | | | | | | | | | |
| Median | 2.0 | 2.0 | 2.50 | 2.75 | 2.0 | 2.0 | 2.0 | 2.0 | 2.25 | 2.0 | 1.50 | 2.0 |
| 75^{th} | 3.0 | 3.0 | 3.50 | 3.38 | 3.0 | 3.0 | 2.50 | 3.0 | 3.50 | 3.0 | 3.0 | 3.38 |
| percentile | | | | | | | | | | | | |
| Minimum | 0 | 0 | 0 | 0 | 0 | 0 | 0 | 0 | 0 | 0 | 0 | 0 |
| Maximum | 4.0 | 8.0 | 4.50 | 5.0 | 4.0 | 4.0 | 5.0 | 5.0 | 5.0 | 6.0 | 5.0 | 5.0 |

TABLE 9

| | Mean value of the wheal (cm) 20 to 60 min after injection | | | | | | | | | | | |
|---|---|---|---|---|---|---|---|---|---|---|---|---|
| | Visit 2 | | Visit 3 | | Visit 4 | | Visit 5 | | Visit 6 | | Visit 7 | |
| | Inj. | Inj. | Inj. | Inj. | Inj. | Inj. | Inj. | Inj. | Inj. | Inj. | Inj. | Inj. |
| | 1 | 2 | 3 | 4 | 5 | 6 | 7 | 8 | 9 | 10 | 11 | 12 |
| Valid | 44 | 44 | 44 | 44 | 43 | 42 | 42 | 42 | 40 | 40 | 37 | 36 |
| Missing | 0 | 0 | 0 | 0 | 1 | 2 | 2 | 2 | 4 | 4 | 7 | 8 |
| Mean | 0.36 | 0.43 | 0.56 | 0.52 | 0.49 | 0.54 | 0.52 | 0.51 | 0.58 | 0.54 | 0.56 | 0.50 |
| Standard | 0.30 | 0.56 | 0.28 | 0.17 | 0.26 | 0.31 | 0.32 | 0.36 | 0.53 | 0.50 | 0.56 | 0.53 |
| deviation | | | | | | | | | | | | |
| 25^{th} | 0 | 0 | 0.4 | 0.4 | 0.3 | 0.4 | 0.3 | 0.4 | 0.3 | 0.3 | 0.3 | 0.3 |
| percentile | | | | | | | | | | | | |
| Median | 0.4 | 0.4 | 0.5 | 0.5 | 0.5 | 0.5 | 0.5 | 0.45 | 0.4 | 0.4 | 0.4 | 0.4 |
| 75^{th} | 0.58 | 0.5 | 0.6 | 0.68 | 0.5 | 0.53 | 0.53 | 0.6 | 0.5 | 0.5 | 0.5 | 0.5 |
| percentile | | | | | | | | | | | | |
| Minimum | 0 | 0 | 0.2 | 0.2 | 0 | 0 | 0 | 0 | 0 | 0.2 | 0.2 | 0.2 |
| Maximum | 1.0 | 3.5 | 2.0 | 1.0 | 1.50 | 2.0 | 2.0 | 2.50 | 2.50 | 3.0 | 3.0 | 3.50 |

Additionally, the typical wheal reactions were below 1 cm. According to guidelines, adaptation of treatment is required when the local reaction reaches the limit of about 5 cm. In the present study, the maximum value observed for a wheal was 3.5 cm.

Especially the wheal diameter is a good indication of systemic reactions.

Immediate allergic systemic reaction emerging within 30 minutes after injection were reported in few patients, the reactions were graded in accordance to the recommendations of AWMF for the management of these reactions (Ring, J., Akuttherapie anaphylaktischer Reaktionen. Leitlinie der Deutschen Gesellschaft für Allergologie und klinische Immunologie (DGAKI), des Ärzteverbandes Deutscher Allergologen (ÄDA), der Gesellschaft für Pädiatrische Allergologie und Umweltmedizin (GPA) und der Deutschen Akademie für Allergologie und Umweltmedizin (DAAU). Allergo J 2007, 16: p. 420-434). Immediate allergic systemic reactions of grade I (mild) were reported in 2 patients (3.3% of patients) and 5 immediate allergic systemic reaction of grade II (moderate) were reported in 4 patients (6.6% of patients). These frequencies are lower than the frequency reported in literature (22% for grade II systemic reactions in a meta analysis of Calderon M A, Alves B, Jacobson M, Hurwitz B, Sheikh A, Durham S Allergen injection immunotherapy for seasonal allergic rhinitis (Review) The Cochrane Library 2007, Issue 1).

### CONCLUSION

The treatment was well-tolerated and highly efficient.

All references cited herein are incorporated by reference to the full extent to which the incorporation is not inconsistent with the express teachings herein.

### Comparative Study

To compare the safety of the preparation comprising mannitol/trehalose to technical phosphate buffer saline preparations. This comparative preparation comprised
100 μg/ml pollen peptides as used in the first study
0.35 mg/ml Phosphate
0.56 mg/ml NaCl
0.01 mg/ml KCl.

The studies differed in that the product had a different formulation and each treatment was splitted into two injections, i.e. more allergens was applied.

TABLE 10

| | Mean wheal diameters (cm) reported 8 hours after injection | | | | | | | | | | | | |
|---|---|---|---|---|---|---|---|---|---|---|---|---|---|
| | | Visit 1 | | Visit 2 | | Visit 3 | | Visit 4 | | Visit 5 | | Visit 6 | |
| | | Inj. | Inj. | Inj. | Inj. | Inj. | Inj. | Inj. | Inj. | Inj. | Inj. | Inj. | Inj. |
| | | 1 | 2 | 1 | 2 | 1 | 2 | 1 | 2 | 1 | 2 | 1 | 2 |
| Comparative | Daily | 5 | | 10 | | 20 | | 50 | | 50 | | | |
| composition | dose (μg)* | | | | | | | | | | | | |
| | N | 9 | | 9 | | 9 | | 9 | | 9 | | | |
| | Mean | 3.56 | | 3.33 | | 2.39 | | 2.72 | | 2.11 | | | |
| | Median | 4.0 | | 5.0 | | 2.0 | | 2.0 | | 2.0 | | | |
| | Minimum | 0.0 | | 0.0 | | 0.0 | | 0.0 | | 0.0 | | | |
| | Maximum | 12.0 | | 9.0 | | 6.5 | | 7.5 | | 7.0 | | | |
| Inventive | Daily | 5 | 5 | 10 | 10 | 20 | 20 | 40 | 40 | 70 | 70 | 100 | 100 |
| composition | dose (μg)* | | | | | | | | | | | | |
| | N | 59 | 59 | 57 | 57 | 56 | 56 | 55 | 55 | 55 | 55 | 52 | 52 |
| | Mean | 0.41 | 0.50 | 0.62 | 0.62 | 0.57 | 0.50 | 0.63 | 0.69 | 0.51 | 0.51 | 0.59 | 0.75 |
| | Median | 0.00 | 0.00 | 0.00 | 0.00 | 0.00 | 0.00 | 0.00 | 0.00 | 0.00 | 0.00 | 0.00 | 0.00 |
| | Minimum | 0.0 | 0.0 | 0.0 | 0.0 | 0.0 | 0.0 | 0.0 | 0.0 | 0.0 | 0.0 | 0.0 | 0.0 |
| | Maximum | 4.8 | 6.8 | 6.5 | 7.5 | 7.8 | 7.3 | 9.5 | 9.0 | 7.3 | 6.3 | 7.0 | 7.0 |

*theoretical daily dose; some patients may have received the next lowest dose if large reactions had been previously observed

Unexpectedly the use of the trehalose/mannitol composition resulted in a reduction of wheal diameter.

1. A pharmaceutical preparation comprising
10 to 200 μg/ml of fragments of an antigenic structure which induces allergic reaction;
2 to 6% (w/v) mannitol;
0.5 to 2% (w/v) trehalose; and
water,
wherein said preparation essentially does not comprise heat shock proteins.

2. The pharmaceutical preparation of claim 1, wherein the concentration of the fragments of an antigenic structure which induce allergic reaction is 50 to 200 μg/ml.

3. The pharmaceutical preparation of claim 1 further comprising buffering agents, preferably phosphate buffer.

4. The pharmaceutical preparation of claim 1, wherein the pharmaceutical preparation is in a form for subcutaneous injection.

5. The pharmaceutical preparation of claim 1, wherein the amount of heat shock proteins is less than 1 μg/ml, preferably less than 0.5 ng/ml.

6. The pharmaceutical preparation of claim 1, wherein the fragments of an antigenic structure are hydrolyzed allergens, preferably prepared by enzymatic hydrolysis of the antigenic structure.

7. The pharmaceutical preparation of claim 1, wherein at least 70% by weight of the fragments are between 1,000 and 10,000 Da.

8. The pharmaceutical preparation of claim 6, wherein the hydrolyzed allergens are obtained by a method comprising the steps of
a) extracting a natural source of allergens comprising allergenic proteins to form an extract,
b) purifying of said extract to remove non-protein components to form a purified extract
c) denaturing said purified extract to form a purified denatured extract,
d) refining the purified denatured extract to remove impurities to form a refined denatured extract,
e) hydrolyzing a denatured allergen to form the hydrolyzed allergen,
f) purifying said hydrolyzed allergen to remove peptides with a molecular weight above 10,000 Da and below 1,000 Da in order to obtain a purified hydrolysate where 70%, more preferably 80%, of the peptides are between 10,000 Da and 1,000 Da
said purified denatured extract comprising proteins, wherein the most abundant (w/w) proteins, forming together at least 60% (w/w) of all proteins, are at least two proteins, and all proteins represent at least 60% (w/w) of the dry weight of the purified denatured extract.

9. The pharmaceutical preparation of claim 6, wherein the hydrolyzed allergens are obtained by a method comprising the steps of:
a) extracting a source of allergens comprising allergenic proteins to form an extract,
b) purifying the extract to remove non-protein components to form a purified extract,
c) denaturing the purified extract with a first denaturing agent to form a purified denatured extract,
d) refining the purified denatured extract to remove impurities to form a refined denatured extract,
e) denaturing the refined denatured extract with a second denaturing agent to form denatured allergen mixture, and
f) hydrolyzing the denatured allergen mixture to form hydrolyzed allergens.

10. The pharmaceutical preparation of claim 1, wherein the antigenic structures are milk allergens, venom allergens, egg allergens, weed allergens, grass allergens, grass pollen antigens, tree allergens, shrub allergens, flower allergens, grain allergens, fungi allergens, fruit allergens, berry allergens, nut allergens, seed allergens, bean allergens fish allergens, shellfish allergens, meat allergens, spices allergens, insect allergens, mite allergens, animal allergens, animal dander allergens and allergens of Hevea brasiliensis.

11. A preparation of claim 1, wherein the preparation is for use in a treatment of allergy comprising at least two injections in a patient at different time points, preferably wherein the preparation is for use in a treatment comprising of 2 to 20 injections with increasing amounts of the preparation.

12. A vial or an application device comprising 0.2 to 1.5 ml of the pharmaceuticals preparation of claim 1.

13. The application device according to claim 12, wherein the application device is a syringe.

14. A kit comprising 2 to 30 vials or application devices of claim 12.

15. A method of treating allergy comprising administering to a patient by a subcutaneous injection, a cumulated dose of 40 μg to 1000 μs of fragments of an antigenic structure in one or more injection sessions using the pharmaceutical preparation of claim 1.

16. The method according to claim 15, wherein injection sessions are performed in intervals of 3 to 10 days.

17. The method according to claim 15, wherein two subcutaneous injections are performed in the same injection sessions at different loci of the patient's body.

18. The method according to claim 17, wherein the two different loci are on the arms of a patient.

19. The method according to claim 15, wherein the treatment is repeated every year.
